(19) United States
(12) Patent Application Publication
(10) Pub. No.: US 2014/0120189 A1
(43) Pub. Date: May 1, 2014

(54) CELL REGENERATING ANTIOXIDANT

(71) Applicants: Anant Arora, Kolkata (IN); Gunjan Aggarwala, Kolkata (IN)

(72) Inventors: Anant Arora, Kolkata (IN); Gunjan Aggarwala, Kolkata (IN)

(21) Appl. No.: 14/128,482

(22) PCT Filed: Jun. 25, 2012
(86) PCT No.: PCT/IN2012/000449

§ 371 Date: Dec. 20, 2013

(30) Foreign Application Priority Data
Jul. 8, 2011 (IN) 917/KOL/2011

(51) Int. Cl. A61K 36/48; A61K 36/42; A61K 36/185

(52) U.S. Cl. CPC A61K 36/48; A61K 36/185; A61K 36/42
USPC 424/758; 424/725

(57) ABSTRACT

The present invention relates to cell regenerating antioxidant composition and the process for preparing the same made from commonly available flowers and fruits which includes banana flowers, cucumber fruits and chickpea to regenerate and repair the cells and tissues within the body. The cell regenerating antioxidant composition made from commonly available flowers and fruits to regenerate and repairs the genetic structure of the cells producing matured adult blood cells by the bone marrow within the body, having right amount of growth factors and blood fuels to be supplied to genetically weak adult cells and tissues thus eliminating major genetic disorders and other cell related disorders through the process of adult cell regeneration. Product to regenerate the bone marrow cells within the body to produce and supply matured adult blood cells with right amount of growth factors and blood fuels for genetically weak adult cells and tissues with the body curing cell disorders and genetic disorders completely.

### FIELD OF THE INVENTION

The present invention relates to a cell regenerating antioxidant composition and the process for preparing the said composition made from commonly available flowers and fruits, which includes banana flowers, cucumber fruits and chickpea, the product repairs and regenerates the cells and the tissues within the body. The cell regenerating antioxidant composition of the present invention helps to regenerate and repair Deoxyribonucleic acid and the genetic structure of the cells within the body, hence eliminating the major genetic disorders and other cell disorders. The cell regenerating antioxidant composition of the present invention regenerates the cells within the body by increasing the magnitude and effectiveness as well as maintaining the balance of growth factors produced in the bone marrow cells and transported to cells within the body through hemoglobin. Particularly, the present invention relates to a cell regenerating antioxidant composition and preparing the same to regenerate and to repair the genetic structure of the bone marrow cells producing matured adult blood cells within the body, having right amount of growth factors and blood fuels for genetically weak cells and tissues thus eliminating major genetic disorders and other cell related disorders. The cell regenerating antioxidant composition cures major genetic cell disorders and other cell disorders either through regeneration as well as replacement of damaged adult cells by a new one depending upon the damage to the cells and tissues.

### BACKGROUND OF THE INVENTION

Cell and tissue damage is a major problem faced by all individuals worldwide and can occur at any age in the life of mankind, could be genetic or environmental starting from birth or an accident, injury etc. to various chronic cell diseases and cell disorders. The drugs and products used currently for mitigating the symptoms of cell and tissue damage in several disorders and diseases have a number of side effects. Surgery however is an option but it can have severe complications.

The present invention relates to cell regenerating antioxidant composition and preparation of the composition thereof made from commonly available flowers and fruits, which includes banana flowers, cucumber fruits and chickpea, which is an oral cell regenerating antioxidant composition and is able to regenerate cells and tissues within the body. The product is used to repair cells and stop the Deoxyribonucleic acid (herein after referred as ‘DNA’) damage completely within the cells further leading to regenerating the adult cells within the body and curing the medical condition. The product stabilizes the genetic structure of the cells within the body, not letting oxidation reactions to take place within the cells and tissues, moreover it helps the bone marrow to produce new blood cells with regeneration ability so as to repairs and regenerates the damaged cell structure by balancing the production and flow growth factors in the blood platelets and other growth factors within the hemoglobin, thus reducing the size of progressive and benign tumors, cysts and the like and hence the product serves for several chronic cell disorders as well as major genetic disorder. The antioxidant nature of the product does not allow the disease to spread and the regenerating property of the product regenerates the cells thus eliminating the disease and its cause altogether.

There are several cell related disorders as well as several genetic disorders faced worldwide which include diseases such as bronchial asthma, cysts, progressive and benign tumors, ulcers, paralysis due to stroke or trauma, coma, hysteria, cancer all kinds (which include primary as well as secondary, where rapid division of cells start to take place), plural effusion from secondary cancer, elevated PSA (prostate specific antigen), bleeding piles, cirrhosis of liver, thalassemia, inflammation, pain as a result of injury from accidents, pus from ear, chronic white discharge (likoria) and the like. Thus this product acts as a cell regenerating antioxidant for the above stated cell disorders and genetic disorders.

There are different modes of treatments which may include medicines, antibiotics and surgery for different diseases but not cure. Cell regenerating antioxidant made as a result of the present invention is a product not to treat but to cure cell related disorders and genetic diseases.

Taking cancer as an example we know that cancer is a major disease growing worldwide as we know Cancer (malignant neoplasm) is a large, heterogeneous class of diseases in which a cell or a group of cells (tissue) display uncontrolled growth, invasion that intrudes upon and destroys adjacent tissues, and often metastasis takes place more so after surgery or biopsy, wherein the tumour cells spread to other locations in the body via the lymphatic system or through the bloodstream. These three malignant properties of cancer differentiate malignant tumours from benign tumours, which do not grow uncontrollably, directly invade locally, or metastasize to regional lymph nodes or distant body sites like brain, bone, liver, or other organs.

Presence of cancer can be suspected on the basis of symptoms, or findings on radiology. Definitive diagnosis of cancer, however, requires the microscopic examination of a biopsy specimen. Most cancers can be treated. Possible treatments include chemotherapy, radiotherapy and surgery. The present invention relates to a product which is a Cell regenerating antioxidant that not only stops the spread of disease making the disease inactive, and more importantly regenerates the cells and tissues by proving the right amount of growth factors, blood fuels, immunity to the cells and tissues within the body, eliminating the rapid uncontrolled outgrowth of abnormally behaving adult cells thus stopping and stabilizing the cell structure through the process of regeneration of the adult cells and tissues, making the cells and tissues normal. As a result further invasion and intrusions are stopped completely inactivating the medical condition thus it opens a new window in the treatment of cancer making it an anti-cancer product. It may be administrated with or without chemo therapy and it neutralizes the side effects of chemo therapy as well and is beneficial in all types of cancers as its major function is to repair and regenerate the abnormal growing cells within the body through the process of regeneration of cells and tissues within the body.

The document RU 237291 discloses an antioxidant remedy by dense extract of hop leaves (Humulus Lupulus L.). The application of the invention extends scope of use of herbal remedies as an antioxidant for prevention and treatment of free radical pathologies.

The document KR 20090092898 discloses a cosmetic composition containing herbal extracts having excellent skin whitening, anti-oxidation, and anti-inflammation is provided to protect skin from the sensitive stress and suppress aging. A cosmetic composition having the effects of skin whitening, anti-oxidation, and anti-inflammation comprises caryophylli Flos, santali alba Lignum, nardostachyos Rhizoma, aquilariae Lignum, foeniculi Fructus, agastachis Herba, hoelen, bletillae Rhizoma, paeonia japonica, ginkgo Semen, ginseng Radix alba, fructus Tribuli Terrestris, ampelopsis Radix. The mixture of herbal extracts is produced by heating at 80˜100 DEG C. for 30 minutes to one hour and adding purified water and low alcohol in 1:1˜5.

The document FR 2905827 discloses an antioxidant additive for incorporation into a food, food complement or food supplement for animal, preferably a ruminant, comprises a herbal extract comprising extracts of artichoke, rosemary, tagete, turmeric extract, extract of citrus fruits, preferably grapefruit, nettle, garlic, grape, milk-thistle, andrographis, green tea and tomato. Antioxidant additive for incorporation into a food, food complement or food supplement for animal, preferably a ruminant, comprises a herbal extract comprising extracts of artichoke (Cynara scolymus L.), rosemary (Rosmarinus officinalis L.), tagete (Tagetes spp. (T. minuta, T. erecta)), turmeric extract, (Curcuma longa L), extract of citrus fruits (Citrus), preferably grapefruit, nettle (Urtica dioica), garlic (Allium sativum), grape (Vinis vitifera), milk-thistle (Silybum marianum (L.) Asteraceae), andrographis (Andrographis paniculata), green tea (Camellia sinensis, Camellia thea Link./Thea sinensis L.), and tomato (Solanum lycopersicum L., Lycopersicum esculentum Mill).

The document UA 66499 discloses a herbal remedy possessing antioxidant and hypolipidemic activity comprises the extracts of stevia leaves and the petals of the chrysanthemum.

The document KR 20040034897 discloses an antioxidant composition containing an extract of herbal plants such as pine needles, Artemisia lwayomogi, Saururus chinensis Baill. and Rubus coranus Miguel is provided. It has better antioxidant properties than ascorbate and BHA known as antioxidants and thus improves food quality when applied to various foods.

The document CN 101985002 discloses a medicinal composition for treating cancer. At present, when the cancer and malignant tumor are treated, surgical operation and radiotherapy and chemotherapy are matched for the treatment. The radiotherapy and the chemotherapy can kill cancer cells, but during the treatment, normal tissue cells are damaged, gastrointestinal reaction, bone marrow suppression and hepatorenal and cardiac function damage are induced, the bodies of patients are more weakened and the further treatment hardly can be performed, and the conventional chemical medicine treatment method cannot prevent the cancer cells from transferring and has high toxic and side effects. The invention provides a natural Chinese herbal medicinal composition for treating cancer, which consists of dark plum fruit, white paeony root, dry vine root, twotooth achyranthes root, the tail part of Chinese angelica, cortex acanthopanacis, great burdock achene and the like. A pharmacological experiment proves that: the medicinal composition can remarkably inhibit the growth of mice hepatoma H22, does not remarkably change the weight of the mice and has the obvious effect on and low toxicity to the growth of a mice transplantation hepatoma H22 cell strain.

The document CN 101524488 discloses a preparation method of compound bamboo leave flavone dripping pill, belonging to the technical field of traditional Chinese medicine preparation. In the dripping pill preparation, Chinese herbal medicine bamboo leaves and mimosa are used as raw materials, a flavone extractive is obtained by extracting according to a certain method, and then the flavone extractive and pharmaceutical accessories used as matrix are processed by a certain method in proportion to prepare the final product. The dripping pill has the characteristics of high bioavailability, quick dissolution time, fast absorption by oral taking, high content of effective ingredients, accurate dosage, and convenient taking and carrying; the invention also has the functions of anti-lipid overoxidation, free radical removal, blood fat and cholesterolemia reduction, coronary blood flow increase, and myocardial ischemia melioration, and can be used for resisting oxidation, resisting aging, lowering blood fat, preventing and curing arteriosclerosis, cerebral ischemia, miocardial infarction and other diseases as well as the adjuvant therapy for cancer.

The document US 2009232786 discloses a therapeutic herbal composition comprising Saw Palmetto, Bromelain, Willow Herb, Grape Seed Complex, Wild Rosella, Liquorice, Passionfruit Seed and Selenium Yeast is claimed. The composition may be used as a general health promotant but is also useful as a treatment of cancer and inflammation. Prostate cancer and hyperplasia may be particularly beneficially addressed with the composition. The invention extends to a method of treatment using a composition as described.

The document WO 2004112692 discloses a therapeutic herbal composition is beneficial for enhancing the immune system and treating such a diverse range of diseases as AIDS, SARS, immune disorders, lymes disease, rheumatic arthritis, cancer. It is also useful as a general blood tonic. This composition includes the synergistic combination of Boswelia carterii stem resin, Styrax benzoin stem resin, Cinnamomum zeylanicum bark, Curcuma zedoaria root, Syzygium aromaticum fruit, Nardostachys chinensis root, Betula alba bark, Impatiens balsamina bark, Costus spicatus root, Allilum sativum bulb and Cyperus rotundus root. In addition, this composition may further comprise Hyssopus officinalis.

The present invention relates to a cell regenerating antioxidant composition so as to regenerate the cells and tissues within a body through commonly available flowers and fruits, which includes banana flowers, cucumber fruits and chickpea, where its major function is to regenerate and repairs the genetic structure of the bone marrow cells producing matured adult blood cells within the body so as to pass through its membrane, having right amount of growth factors and blood fuels for genetically week cells and tissues thus eliminating major genetic disorders and other cell related disorders. The present invention also relates to its preparation of the composition to regenerate bone marrow cells within the body to produce and supply matured adult blood cells with right amount of growth factors and blood fuels for genetically weak adult cells and tissues within the body curing cell and genetic disorders within the body. Regeneration herein is the process through with a lost or damaged part of a cell or a tissue within the body of an organism is restored to its original function.

The following invention also acts as an antioxidant. Where oxidation is the process through which free radicals are produced within the body, in turn these free radicals can start chain reactions that damage DNA within the cell due to which major cell and genetic disorder take place. Antioxidant nature of the product terminate these chain reactions by removing free radical intermediates and regenerative nature of the product increasing the resistance of the cells so as to withstand the damages caused by those oxidation chain reactions, and inhibit other oxidation reactions within the DNA of the cells, thus the regenerative nature of the product regenerates the DNA within, more over increases the resistance of cells against oxidation by enhancing the immunity of cells against oxidation. On the other hand production of balanced amount of growth factors by bone marrow cells and supplied through hemoglobin makes the above possible and so the regeneration process restore the already damaged cells and tissues to their normal function. In most of the cases we have seen that oxidation or cell damage occurs due to inadequate amount of growth factor within the cells which makes the cells immunity against oxidation negligible. Once the cell regenerative antioxidant is administrated it provides the balanced and right amount of growth factors to the cells and tissues within the body as they can increase immunity of cells and tissues to fight oxidation damage and eliminating it all together by regenerating the adult cells restoring the cell and tissue function to normally.

The present invention relates to a cell regenerating antioxidant prepared from commonly available flowers and fruits which includes banana flowers, cucumber fruits, chickpea and the like so as to regenerate the cells and the tissues within the body curing several cell disorders as well as genetic disorders as it repairs and minimizes DNA damage and oxidation of cells and tissues in the body. DNA is a nucleic acid that contains genetic instructions used in the development and functioning of all known living organism, when oxidation takes place it damages the DNA and it cannot be repaired or regenerated but the damage can be minimized in modern science but though this cell regenerating antioxidant the adult damaged cells can be repaired and regenerated and even replaced by new adult cells eliminating the genetic disease and cell disorder completely over a short span of time. We know that the DNA is transferred from parent to the child and so are the genetic disorders since the child receives genetically weaker cells there arises the genetic disorder, the product cures genetic disorders through its cell regenerating ability hence major genetic disorders are eliminated from one generation to the other with the use of cell regenerating antioxidant. Apart from the genetic transfers of disorders from parent to child the DNA can be damaged by many sorts of mutagens, these mutagens include oxidizing agents, alkylating agents, accidents, accidents and also high energy electromagnetic radiations such as ultraviolet. The product formed as a result of the present invention is a cell regenerating antioxidant it does not allow oxidation reactions to take place within the cells and more over it regenerates the cells and tissues by stimulating the right amount of growth factors in bone marrow and blood platelets so as to be transferred to the damaged and genetically weaker cells within the body through blood.

### OBJECT OF THE INVENTION

The major objective of the invention is to provide cell regenerating antioxidant product and the preparation of the cell regenerating antioxidant made from commonly available flowers and fruits that includes banana flowers, cucumber fruits and chickpea to regenerate adult cells and tissues within the body so as to cure and treat several cell related disorders as well as several genetic disorders through oral intake which include diseases such as bronchial asthma, cysts, progressive tumors, benign tumors, ulcers, paralysis due to stroke or trauma, coma, hysteria, cancer of all kinds (which include primary as well as secondary), plural effusion from secondary cancer, elevated PSA, bleeding piles, cirrhosis of liver, thalassemia, inflammation, pain cure as a result of injury from accidents, puss from ear, chronic white discharge (likoria).

It is one of the objects of the invention to provide a cell regenerating antioxidant composition wherein the cell regenerating antioxidant comprises of 97% by weight banana flowers, 2.5% by weight cumber fruits and 0.5% by weight chickpea.

It is one of the objects of the invention to provide cure for pain within hours and minutes from injury or accident through cell regeneration.

The most important objective of the present invention is to provide an anti cancer product.

It is another object of the present invention to provide a product to neutralize the side effects of chemo therapy, by increasing the resistance of human body.

It is an object of the present invention to provide longevity and healthy life to all individuals as it can be administered with or without disease, it regenerates and repairs the damaged cells and tissues stabilizing the metabolism, and doesn't allow oxidation reactions to take place therefore increasing longevity and life span.

It is an another object of the invention is to provide a product as a cell regenerative antioxidant made from commonly available flowers and fruits, which includes banana flowers, cucumber fruits and chickpea that repairs and regenerate the genetically weak bone marrow cells so as to produce healthy hemoglobin with balanced growth factors and proper immunity against cell and DNA damage. The production and supply of healthy hemoglobin through bone marrow cells and blood platelets helps in curing major genetic disorders and other cell related disorders.

It is another object of the invention to provide a cell regenerating antioxidant which is used to stabilize the metabolism within the body.

It is yet another objective of the present invention to provide the most powerful new age food for the adult and embryonic cells and tissues so as to stop cell and tissue damage within the body.

It is therefore one of the objectives of the present invention to provide a cell regenerative antioxidant which can take the form of powder, granules, tablets, capsules, suspension, injection, syrup, tincture and adhesive plaster.

It is yet another objective of the present invention to prepare the cell regenerating antioxidant composition made from commonly available flowers and fruits that includes banana flowers, cucumber fruits and chickpea.

It is one of the objects of the invention to prepare a cell regenerating antioxidant composition wherein the cell regenerating antioxidant comprises of 97% by weight banana flowers, 2.5% by weight cumber fruits and 0.5% by weight chickpea.

It is yet another object of the present invention to provide a cell regenerative antioxidant that can be administered using numerous carriers including butter, milk, starch, sugar, cream, honey, vegetable oils, edible oils, fats, gums, glycols, water and other known excipients , it works best with white butter or yellow butter.

### SUMMARY OF THE INVENTION

The above and other objects of the invention are achieved by providing a regenerating antioxidant in from of standardized extracts made from commonly available flowers and fruits preferable banana flower and cucumber fruit and chickpea so as to regenerate cells and tissues within the body.

The following invention relates to a cell regenerating antioxidant made from commonly available flowers and fruits (preferably banana flower, cucumber fruit and chickpea) below 100° C. with standardized extraction processes so as to get a product which regenerates the cells and tissues within a body. It helps to stabilize the metabolism of the body, provide the balanced amount of production and supply of growth factors to cells and tissues; it not only antioxidizes but majorly regenerates and repairs the cells and the tissues as it reduces the size of progressive tumors, benign tumors and cysts minimizing DNA damage within the cells and regenerating it so as to restore the cell to its normal function.

The cell regenerating antioxidant can take various forms including powder, granules, pills, tablets, capsules, suspension, injection, syrup, tincture or adhesive plaster. It can be administered using numerous carriers including starch, milk, sugar, honey, butter, cream, vegetable oils, edible oils and fats, gums, glycols water and other excipients but works best with white or yellow butter.

The cell regenerating antioxidant is a product that can be administered to any individual who is suffering from genetic disorders or cell related disorders or cell and tissue damage or even to healthy individuals suffering from no problems, since it is a cell regenerating antioxidant it prolongs the life span of individuals and does not let oxidation reactions to take place within the body more over it regenerates the existing damaged cells and tissues thus increasing their life span and longevity with a healthy life. It is a no side effect product since it is made from commonly available flowers and fruits therefore it can be administered to any individual with or without disease. In case of a cell damage or genetic disorder within the cells and tissues of the body, the product regenerates such weak cells and tissues within the body and remove the cause of the disease altogether.

The present invention provides a product that is made from banana flower, cucumber fruit and chickpea, or banana flower, stem of the banana tree, cucumber fruit and chickpea, so as to get the cell regenerating antioxidant.

The present invention provides a product made from banana flower, cucumber fruit and chickpea or the combination banana flower, stem of banana plant cucumber fruit and chickpea using standardized extraction techniques below 100° C. so as to get the pulverized dark brown mass which is powdered as cell regenerate antioxidant to regenerate cells and tissues within the body through oral intake in diseases related to genetic as well as cell disorders which are bronchial asthma, pain cure as a result of injury from accidents, progressive tumors, benign tumors, cysts, ulcers, paralysis due to stroke or trauma, coma, hysteria, all kinds of cancers (which include primary as well as secondary), plural effusion from secondary cancer and spread of cancer in secondary stage, reducing elevated PSA, bleeding piles, cirrhosis of liver, thalassemia, inflammation, puss from ear, chronic white discharge (likoria) and the like.

### DETAILED DESCRIPTION OF THE INVENTION

Cell and tissue damage is a major problem faced by all individuals and can occur at any age in the life of mankind, starting from genetically weak cells from parent to child, or environmental factors like an accident to various chronic diseases and disorders. The drugs and products used currently for mitigating the symptoms of cell and tissue damage in several disorders and diseases have a number of side effects. Surgery however is an option but it can have severe complications.

Today tissue damages happens at any stage of life in mankind due to genetic factors, cell and tissue damage due to accidents, injury, environment factors and the like or through outgrowth of cells and tissues as a result of DNA damage and oxidation damage due to the presence of genetically weak cells and due to other various reasons within the body, these factors may cause diseases like bronchial asthma, pain as a result of injury from accidents, cysts, progressive tumors, benign tumors, ulcers, paralysis due to stroke or trauma, coma, hysteria, cancer of all kinds (which include primary as well as secondary cancers, where cells start to grow abnormally anywhere within the body), plural effusion from secondary cancer, Elevated prostate specific antigen, bleeding piles, cirrhosis of liver, thalassemia, inflammation, puss from ear, chronic white discharge (likoria) and other cell related medical conditions the product hence formed is a cure as well as a treatment for the above mentioned disorders and diseases.

The cell regenerating antioxidant made from commonly available flowers and fruits to regenerate and repairs the genetic structure of the bone marrow cells producing matured adult blood cells within the body, having right amount of growth factors and blood fuels to be supplied to genetically week cells and tissues thus eliminating major genetic disorders and other cell related disorders through the process of adult cell regeneration. Product to regenerate the bone marrow cells within the body to produce and supply matured adult blood cells with right amount of growth factors and blood fuels for genetically weak adult cells and tissues with the body curing cell disorders and genetic disorders completely.

Taking an example of accidents or injuries which can happen to any individual in normal course of life, through automobiles, slipping, collision, injury to athletes which leads to ischemia as the cells and tissue get damaged, this happens because of the restriction of blood supply due to oxidation damage within blood vessels, this may lead to dysfunction of the cells and tissues of the given part of the body. Ischemia is basically absolute or temporary shortage of blood supply to the cells and tissues within the body, or to the cells and tissues of the organs within the body, which includes shortage of oxygen, glucose, and blood borne fuels and a series of balanced growth factors to the cells and tissues facing damage within the body. A relative shortage means the mismatch of blood supply (oxygen/fuel delivery) and blood required for adequate metabolism of tissue. Ischemia results in cells and tissue damage because of a lack of oxygen and nutrients.

The cell regenerating antioxidant must be administrated within 20minutes from injury, or accident, or conditions of ischemia for best results. Once administrated the product regenerates the damaged cells and tissues helps to stabilize the metabolic rate and maintain adequate flow of blood in the affected area with the adequate supply of oxygen, glucose and other blood borne fuels to the damaged cells and tissues in the body as it immediately activates the bone marrow cells to produce enough blood having balanced growth factors to regenerate and repair the damaged adult cells in the affected area. By doing to it stops further oxidation damage to the cells and tissues of the affected area as the cells regenerate and become immune against the damage and further the regeneration process continues restoring the cell and tissue function back to normal. Its seen symptoms of pain, blood clots, inflammation and the like seem to neutralize and disappear over a short span of time, results seen within minutes and hours from administration. This regenerative nature of the product makes it a pain cure in case ischemia due to various reasons such as accidents, injury and the like.

Taking cancers as another of the examples we know that the disease is rapidly spreading worldwide, it's a class of disease in which cells or a group of cells or tissues display uncontrolled growth, invasion that intrudes upon and destroys adjacent tissues, and sometimes metastasis, this may further lead to spreading of disease to other locations in the body via lymph or blood. The presence of cancers can be suspected on the basis of symptoms, or findings on radiology. Definitive diagnosis of cancer, however, requires the microscopic examination of a biopsy specimen. Most cancers can be treated. Possible treatments include chemotherapy, radiotherapy and surgery. However chemotherapy, radiotherapy and surgery have their side effects, in case of surgery or fine needle aspiration cytology (FNAC) also known as biopsy, there are maximum chances of spread of cancer within the body through lymph nodes and through blood cells, if the test results are positive, hence chemotherapy is given to the patient. Chemo therapy has its side effects because it cannot differentiate between cancer cells (malignant cells) and normal cells hence it kills all the cells, radiation therapy on the other hand is another alternative but it also damages the cells around which may not be malignant hence it also has a series of side effects known to modern science like chemotherapy.

The cell regenerating antioxidant should be administrated at least 4 weeks before the FNSE or surgery so that it neutralizes its side effects (spread of disease) and in turn reduces the progressive tumors considerably. In case it's a benign tumor, progressive tumor or a cyst, when administered with cell regenerative antioxidant, the abnormal cells regenerate and reduce its size and intensity and pain caused as a result of tumor cells and tissues disappears. Normalization of cells and tissues take place due to the products regenerative nature and further no oxidation or DNA damage takes place as the product is an antioxidant as well, as we know in certain cases surgery need not be required at all. In case of suspicion of malignancy FNSE or surgery is performed and in case if it show positive test results then doses of the cell regenerator are administrated accordingly. In case a person undergoes chemo therapy the cell regenerating antioxidant can be administrated to regenerate the cells, so that the cells in and around the malignancy have been provided with enough resistance against invasion of non malignant cells by malignant cancer cells and spreading the disease over to a larger area. The cell regenerative antioxidant provides resistance to the non malignant cells in the affected area as well as within the body to avoid spread through lymph nodes or intrusion, its major aspect is that unlike chemotherapy it can differentiate between the malignant cells and non malignant cells and it regenerates only non malignant cells. Providing them enough resistance and growth factors to counter the malignant cells and not allow them to invade and grow. Cell regenerative antioxidant provides enough growth factors to the non malignant cell it helps them to maintain balanced growth and have greater immunity to fight the cancer cells and stop invasion against malignancy, on the whole the products ability is regenerating the uncontrolled growth of adult cells and tissues and more so neutralizing the spread of disease further, thus completely inactivating the medical condition. When cell regenerative antioxidant is administered with chemotherapy it neutralizes the side effects of chemotherapy as it regenerates the non malignant cells so the patients can complete their chemo cycle like healthy individuals. Once the chemo has destroyed the cancer/malignant cells the regenerative property or the product replaces new adult cells in the affected areas through regeneration at a faster rate therein restoring the function of the cells and tissues within the organ or the body to normal inactivating the disease completely.

Cell Regenerative antioxidant in case of progressive tumors, malignant tumors or benign tumors should be administered for a minimum period of 8 months so that blood of the body is replaced 2ce over and blood produced within the body by the bone marrow during the phase is so strong that they it not allow any kind of invasion by malignant cells, or rapid dividing cells or any form of abnormal cells and tissues that could harm elsewhere within the body, gradually the malignant cells tend to fade away and die in the absence of food. In certain cases the cell regenerator could be used up to a period of 12/16 months and over.

Cell regenerating antioxidant provided in the instant invention is a no side effect product and is able to regenerate the cells and tissues within the body. Its antioxidant nature stops the growth of the diseases and disorders of the cells and tissues and primary cell regenerating property restores the cell and tissue outgrowth back to normal over a short span of time. Hence it is able to stop the growth of progressive tumors benign tumors and cysts, and restore the outgrown tumor cell back to its normal function thus not allowing the surgery stage to arise in most circumstances.

The cell regenerating and antioxidant nature of the product formed is able to treat cell and tissue damages through regeneration of adult cells and tissues in diseases like bronchial asthma(cure), reducing and neutralizing progressive tumors, benign tumors and cysts, cure for ulcers, early paralysis due to stroke or trauma, coma, hysteria, cancers of all kinds (which include primary as well as secondary cancers), plural effusion from secondary cancer, reducing the PSA levels, bleeding piles, cirrhosis of liver, thalassemia, inflammation, pain cure as a result of injury from accidents, puss from ear, chronic white discharge (likoria), reducing specific glycoprotein levels in the blood, more over the product is used in maintaining healthy hemoglobin levels thus providing metabolic balance within the body and increasing the resistance against oxidation of cells and tissues.

Cell regenerating antioxidant product formed by the instant invention nullifies the side effects of chemo therapy in cancers relating to cell and tissue (primary and secondary) by increasing the resistance of the non malignant cells and tissues toward cell damage, it regenerates progressive tumors stops the out growth and inactivates the disease. The regenerating nature of the product makes it an anti cancer product since the outgrowth of cells and tissues are restored back to normal and the disease become completely inactive.

The present invention provides a product made from banana flower cucumber fruit and chickpea or stem of the banana plant, banana flower cucumber fruit and chickpea or the combination of all of the above using standardized extraction techniques below 100° C. so as to get the pulverized dark brown mass which is powdered to form the regenerating antioxidant.

The cell regenerating antioxidant formed as a result of standard extraction process from commonly available flowers and fruits preferably the banana flower cucumber fruit and chickpea. Banana flower is brown in color and the cucumber fruit is green in color chickpea is an edible legume high in protein, all three of them are edible and are eaten in India and elsewhere. Banana flower is used to make chop's, cucumber is used as a salad fruit and chickpea is used in several Indian dishes. All of them are safe to eat and easily digested. Available in the local markets throughout the country and India being one of the largest producers of the same

To prepare the cell regenerating antioxidant, banana flower and cucumber fruit are obtained and sorted to discard the ones with poor quality. Then the flowers and fruits are washed and dried by draining away the water completely and leaving in air for some time.

The flowers and fruits as a whole (preferably as a whole), or chopped, crushed or any other standard method are placed in an aluminum or stainless steel vessel and after 5-7 days the flowers and fruits shrink and leave a reactant liquid mixture which is drained out in a similar smaller vessel and chickpea is added to it and soaked for 12 hours later the reactant is reintroduced in the former vessel leaving the chickpea to germinate for next 24-36 hours, once germination starts chickpea is also introduced in the former vessel with the reactant liquid mixture and the shrunk flower and fruits, thereafter various standard extraction techniques are used to get the regenerating antioxidant below 100° C. After getting the distillate, it is dried, powdered, sieved and filtered to get standardized extracts. The product hence made is standardized as per standard specifications of plant extracts.

The product obtained is dark brown pulverized material, which is powered into fine particles in moisture free room below 20° C., the color of the power is dark brown and can be administered in its powered state, granules, pills, tablets, capsules, suspension, injection, syrup, tincture adhesive paste and the like.

The cell regenerative antioxidant should be carefully stored in a cool dry place preferably below 28° C. and should be kept away from direct sunlight and other forms of radiation. The product has its own shelf life and resistance, can be used anytime as has no expiry. Once powdered it can be administered using numerous carriers including butter, milk, starch, sugar, cram, honey, vegetable oils, edible oils, fats, gums, glycols, water and other known excipients, it works best in powdered form with white butter or yellow butter. The dosage of the product ranges from 40 mg up to 3000 mg during the day depending on the cell damage.

### EXAMPLE 1

The banana flowers and cucumber fruits as a whole (preferably as a whole), or chopped, crushed or any other standard method are placed in an aluminium or stainless steel vessel, the vessel is covered with a lid and the lid is filled with water on top to maintain the temperature inside the vessel below 100° C. throughout, the vessel is heated and cooled below 100° C. for a period of 5-7 days and then the reactants of the flower and fruits leave a residue liquid reactant mixture which is drained out and collected in another similar smaller stainless steel or aluminium vessel leaving the flowers and fruits in the former vessel intact but shrunk in size compared to their addition in the beginning of the reaction, this solid shrunk flowers and fruits in the former vessel are stirred (by way of rotating the semi solid substance upside down) after the drain out.

Then chickpea is added to the small vessel where the reactant mixture of flower and fruit in liquid state are collected. Chickpea is soaked in with the liquid reactant mixture of flower and fruit for 12-15 hours. After 12-15 hrs the chickpea is removed from the reactant mixture soaked with chickpea is reintroduced in the former vessel with the solid shrunk flower and fruit, thereafter the processing continues. Meanwhile the chickpea is allowed to germinate over the next 24 to 36 hrs. The objective of the above is to allow chickpea to react and germinate with the liquid reactant mixture of the flower and fruits, as soon as the germination completes chickpea is introduced into the former vessel with the flowers, fruits and its liquid reactant mixture and processing takes place for another 5-7 days and the ingredients are processed below 100° C. covered with a lid filled with water on top.

Meanwhile after 3-4 days from the germinated chickpea addition to the former vessel the excess of residue at the bottom of the vessel in liquid state, the process of distillation (by the process of evaporation and condensation) starts, thereafter the shrunk mixtures of the flowers fruits and chickpea is occasionally stirred (by way of rotating the semi solid substance upside down) over the next 3-4 days during the course of distillation (by the process of evaporation and condensation) till the pulverized dry extracts of the cell regenerating antioxidant is formed. Total process to get the cell regenerating antioxidant takes about 14-16 days depending upon the quantity of raw materials and temperature.

After getting the dry distillate, it is, powdered, sieved and filtered to get standardized extract of cell regenerating antioxidant. The product hence made is standardized as per standard specifications of plant extracts.

The product obtained is dark brown pulverized solid material, which is powered into fine particles in moisture free room below 20° C., the color of the power is dark brown and can be administered in its powered state, granules, pills, tablets, capsules, suspension, injection, syrup, tincture adhesive paste and the like. The quantity of the packaging may range from 40 mg to 1000 mg or from 80 mg to 3000 mg during the day depending upon cell damage.

### EXAMPLE 2

Banana flowers cucumber fruits and chickpea are used to prepare the cell regenerating antioxidant using standard extraction processes below 100° C. The product formed is a pulverized dark brown mass which is powdered to get the cell regenerating antioxidant.

The product when administered on 17 Aug. 2010 to patient with case history as follows. The patient diagnosed with a large 17.3*13.1*14.5 cm sized complex cystic SOL with solid areas and septae in the the abdomino-pelvic region In February 2009. On 28 Feb. 2009 Uterus with right and left sided ovarian cysts was removed biopsy suggested malignant cells. Thereafter 6 cycles of chemotherapy were given over a course of time.

PET SCAN was done on 13 Mar. 2010 after more than one year from the operation and it showed a Large (4*4.8*4.8 cm) mass adjacent to right lobe of liver. Further 3 cycles of chemotherapy were given. Surgery of the same was suggested around 1^{st} week of June, However the USG of the whole abdomen done on 30 May 2010 stated a large heterogeneous SOL of 10*9.5 in the right lobe of the liver and she acquired a urine infection and chest congestion where she had breathing problem.

Further USG of the whole abdomen done on 10 Jun. 2010 suggested hepatic parenchyma to be heterogenous & multiple hypoechoic SOL seen in the right lobe and hepatobiliary channels were not dilate.

On 24 Jun. 2010 500 ml of yellow colored pleural fluid was removed from the right lung of the patent, same was done in respective month i.e. 10^{th} July and August 14^{th} where the right lung had large and massive pleural effusion. The Patient was in the 4^{th} stage of cancer with pleural effusion in right lung and heterogeneous SOL in liver.

Cell regenerating antioxidant was administered 2 days after the drain out on 14 Aug. 2010 i.e from 17 Aug. 2010 and their after the breathing problem reduced considerably within days and chest X Ray were done on 11 Oct. 2010 & 12 Nov. 2010 respectively as there was no further pleural fluid was deposit in and after the last drain out and it showed no presence of the soft tissue seen on 3 Aug. 2010. The water level was same in the lungs as it were left on 14^{th} August after the drain out with mild pleural thickening seen. However the comments in the second X ray done on 12 November, suggests pnemuno nitis with ill defined opacities in lower zones of both lungs.

Further x ray was done on 15 Dec. 2010 suggested mild cardiomegaly seen with right minimal thickening, no active lung parenchymal lung was seen and everything seem normal.

Further digital radiography done on 4 Mar. 2011 suggested no active lung parenchymal lung lesion, costophrenic angles were clear. And the patient was leading a normal life without any breathing problems. No sign of pleural effusion is seen or there has been a need to drain out pleural fluid since the last drain out on 14 Aug. 2010 till date 30 May 2011.

During the same period USG done on 13 Mar. 2010 showed a Sol of 4.8*4.2*5.2 cm with central cystic areas in the right lobe of the liver thereafter on 30 May 2010 a large heterogeneous SOL of 10*9.5 was seen in the right lobe of the liver further USG done on 10 Jun. 2010 showed hepatic parenchyma to be heterogenous & multiple hypoechoic SOL seen in the right lobe and hepatobiliary channels were not dilate.

When the ultrasonography was during the period when the patient was administered with cell regenerating antioxidant on 12 Nov. 2010 no abdominal fluid was seen, liver was normal in size and homogenous in parenchymal echotexture. A cystic sol (74.5 mm*68.2 mm*64.3 mm in size and 171.0 cc in volume) was seen in the right lobe. Another cystic Sol (57.9 mm*52.7 mm*63.9 mm is size and 101.9 cc in volume) was seen infero lateral to the larger one. Impression suggested cystic Sol in liver.

Another USG done on 15 Dec. 2010 described the Sol further with not much comprehensive increase in size and volume where the size varied in segment VII SOL that measured 75*85*73 mm (volume 245 CC) and segment VI SOL measuring 63*66*51 mm (volume 111 cc). Impression suggested Sol likely to be abscess, comples thick walled cystic lesion in pelvis.

Further USG done on 3 Mar. 2011 showed sol measuring 91*76*92 mm=337 cc in the diaphragmatic region and prosterior segment of the liver. Another thick walled cystic sol with low level echos measuring 69*59*88=190 cc seen anteriorly to it. Rest of the liver was homogenous. Impression suggested mild increase in size from previously visualized liver abscesses and pelvic cyst had increased in size relative with previous study.

Further USG done on 2 Apr. 2011 suggested a thick (11 mm) walled cystic Sol seen in the right lobe of the liver having volume of 344 cc and mass of 96*68*100 mm the report suggested that thick walled liver abscess had not decreased from previous report but there was no considerable increase as well.

The patient is leading a normal life till date 29 Jun. 2011 with the change of a large heterogeneous Sol into a homogeneous parenchymal ecotexture to cystic sol to abscess.

In between there was hemorrhage in her pelvic region during April 2011 which was stopped by monitoring the dose of the cell regenerating antioxidant but due to excessive bleeding the CA 125 levels rose considerable, treatment undergoing to monitor CA 125 levels.

### EXAMPLE 3

The cell regenerating antioxidant when administered to several bronchial asthma patients, we see results within 7 days from administration and a cure within a period of 4 months depending on the genetic cell disorder, the bronchial asthma is a genetic disorder, the genetically weak bronchial cells and tissues cause inflammation create a breathing mechanism disorder, once the cell regenerating antioxidant is administrated it provides right amount of growth factors and immunity through the blood cells produced by bone marrow to the genetically weak adult cells and tissues in the bronchial system, increasing the resistance of cell and tissue against damage and oxidation and more importantly, the products cell regenerating property proving balanced growth to the adult cells and tissues in the bronchial system regenerating them, removing inflammation of cells and tissues neutralizing the cause of bronchial asthma as a whole. There after cells and tissues in the bronchial system starts to functions normally with proper amount of immunity against further damage, thus curing bronchial asthma over a short span of time. Cell regenerating antioxidant is a product to cure the genetic disorder of cells in bronchial asthma permanently and provides relief for sinus problem.

### EXAMPLE 4

The cell regenerating antioxidant has been administered to patient suffering from immediate accident, injury, twist of ankle, inflammation, collision, automobile accidents and the like, injury to athletes which leads to ischemia as the cells and tissue get damaged, this happens because of the restriction of blood supply due to oxidation damage due to factors of blood vessels, this may lead to dysfunction of the cells and tissues of the given part of the body. Ischemia is basically absolute or temporary shortage of blood supply to the cells and tissues within the body, or to the cells and tissues of the organs within the body, which includes shortage of oxygen, glucose, and blood borne fuels most importantly series of balanced growth factors to the cells and tissues facing damage within the body. A relative shortage means the mismatch of blood supply (oxygen/fuel delivery) and blood flow for adequate metabolism of cells and tissue. Ischemia results in cells and tissue damage because of a lack of oxygen and nutrients.

The cell regenerating antioxidant when administered within 20 minutes from injury, or accident, or conditions of ischemia acts immediately on the affected area by providing adequate blood flow with right amount of oxygen supply and nutrients for the tissues to survive further oxidation and damage, moreover it enhances the supply of growth factors, immunity and other blood fuels to the affected cells and tissues due to its major cell regeneration property hence regenerating the affected adult cells and tissues within minutes and the pain is not allowed to arise, if pain is sensed it tends to reduce and disappear over a short time frame as a result of adult cell regeneration.

Once administrated the product regenerates the damaged cells and tissues helps to stabilize the metabolic rate and maintain adequate flow of blood in the affected area with the adequate supply of oxygen, glucose and other blood borne fuels to the damaged cells and tissues in the body. By doing to it stops further oxidation damage to the cells and tissues of the affected area more over the cells regenerate and become immune against the any further damage. The regeneration process continues restoring the cell and tissue function back to normal by providing the adequate supply of growth factors to the cells and tissues through blood produced by bone marrow cells, regenerating the affected adult cells and tissue, so we see the symptoms of pain, blood clots, inflammation and the like, seem to neutralize and disappear over a short span of time, results seen within minutes and hours from administration (best results seen within 20 minutes from administration) in some cases depending upon the damages. This cell regenerative nature of the product makes it a pain cure.

### EXAMPLE 5

Cell regenerating antioxidant was administered to patients suffering from early paralysis or early coma, as a result of stroke or trauma we see fast recovery for paralysis patents as well as early coma patients. There are several conditions responsible for a stroke to happen which may include, high blood pressure, hypoglycemia, head injury, brain damages and the like leading to inflammation of cells and tissues in the brain hence restricting blood supply to the connective tissues and cells of the nervous system or the brain. The restriction of blood supply as a result of inflammation, clots, connective cells and tissue damages in the brain and the like does not allow the right amount of blood, oxygen glucose and other blood borne fuels to flow to and from the brain leading to oxidation and further damage of cells and tissues within the brain. A relative shortage of blood flow means the mismatch of blood supply (oxygen/fuel delivery) and blood flow required for adequate survival and proper functioning of tissue and cells in the brain, thus damaging the cells and tissues completely. Once the damages of the connective tissues take place it restricts the flow of instructions from the brain to the other parts of the body paralyzing the specific parts of the body, which could be the right side of the body or left side of the body known as hemiplegia, there could also be symptoms such as facial paralysis, global paralysis throughout the body, and the like.

In several cases is seen that an immediate first aid administration of the cell regenerating antioxidant (within 20 minutes for the first dose for immediate cure) to such patients suffering from stroke or trauma the product immediately regenerates the connective cells and tissues by providing adequate flow of blood, carrying the balanced growth factors, blood borne fuels, oxygen supply, glucose and other factors within so as to increasing the resistance of damaged cells and tissues preventing further oxidation. Mean while the products regenerating property increases the adequate amount of growth factors in the blood supplied to the affected area for cell survival and connective cell and tissue regeneration, neutralizing the cause of paralysis completely, so the conditions of paralysis need not arise at all. Hence within minutes of the first aid of the our cell regenerating antioxidant it is observed that the medical condition caused due to stroke neutralizes, and the patient becomes normal over a short span of time and starts to lead a normal life like before curing the medical condition completely.

### EXAMPLE 6

Cell regenerating antioxidant has been administrated to patients suffering from thalassemia where blood transfusion was done every 20 days, we see results within weeks from administration, as the transfusion process is extended from 20 days to 30 days, to 50 days and so forth and cure for thalassimia is seen once the blood in the body changes 3 times over (8-12 month or more depending upon the extent of the disease). As we know thalassimia is a genetic disorder where in which the body makes an abnormal form of hemoglobin, the protein in red blood cells that carries oxygen to the cells and tissues in the body. The bone marrow produces blood, the cells bone marrow in large bones produces new blood cells, and it is a vital element of the lymphatic system, as it produces lymphocytes and acts to prevent the backflow of lymph. The cells in the bone marrow are genetically weak hence the disorder, we with this product regenerate the adult cells within the bone marrow, and it produces the right amount of matured adult cells so as to pass through bone marrow membrane into the body, with proper immunity and growth factors so that blood count does not fall over and over again thus tackling the thalassimic medical condition and curing the medical condition completely.

### EXAMPLE 7

Cell regenerating antioxidant when administrated to patient suffering from progressive tumors, where biopsy was suggested and the patient started to use the product, we see considerable changes in the medical conditions.

To give a simple example a 72 year old patient where ultrasound of thyroid was done and patient was suffering from enlarged cystic areas of 3 mm and 9 mm in the left lobe of the thyroid respectively and these cystic sol had immense pain in it didn't allow the patient to eat or even talk properly, the neck had swollen up as well, biopsy was suggested. The patient started with the intake of cell regenerator and the pain disappeared within hours of administration of the first dose, due to abnormal adult cell regeneration. The treatment is ongoing and there is no pain to the patient and the cysts seems to neutralize in a short span of time, the patient can now eat and talk like healthy individual which at one point seemed impossible so we can conclude that the abnormality caused as a result of progressive cystic/tumor cells, to regress and become normal as a result makes the regenerative antioxidant an anti cancer product.

Apart from this several men and woman have been administered with cell regenerating anti oxidant, having medical condition of cysts, benign tumors and other forms of progressive tumors and cysts in different parts of the body, such as organs, glands and the like, soon after administration of cell regenerating antioxidant on a regular basis its seen that such tumors, cysts and the like tend to neutralize regenerating the abnormally growing cell back to normal due to its cell regenerative ability. Hence it cures the outgrowth of abnormal cells and tissues making them perform normally like before thus the cell regenerating antioxidant is an anti cancer product.

### EXAMPLE 8

The product when administered to patients suffering from ulcers within the body, peptic duodenal, intestinal and the like its seen that as soon as the regenerative antioxidant is administered to the patients in such medical condition its seen that the cells start to regenerate. The immune mechanism of cells start to function normally after the cell regenerating antioxidant is administered and the inflammation of the weak adult cells seem to disappear The affected abnormal adult cells start to regenerate. Depending upon the spread of disease within weeks the ulcers are no longer present and the cells start to function normally hence curing the medical condition completely.

### EXAMPLE 9

Product when administered to patients suffering from bleeding piles it is noted that the bleeding stops and the pain and inflammation also subsidizes and the abnormal or weak adult cells in the region start to regenerate and perform normally curing the medical condition completely and surgery need not be performed.

### EXAMPLE 10

Product when administered to patients suffering from cirrhosis of liver it is seen that the abnormal cells in the liver start to regenerate and oxidation of cells stops completely, the cell and tissue regeneration process continues depending upon the extent of the disease, over a course of time the medical condition seem to neutralize regenerating the liver cells with proper flow of blood, and making the tissues function normally which makes the patient lead a normal life and perform daily activity like any healthy individual. Once the adult cells and tissues regenerate the patients liver starts to normalize curing the medical condition and surgery need not be performed. Cell regenerating antioxidant is a cure for cirrhosis of liver

### EXAMPLE 11

Product when administrated to several cancer patients for medical conditions such as breast cancers, uterus cancer, ovarian cancer, oral cancer, large bcell cancer, kidney cancer, liver cancer, blood cancers, and all other cell and tissue related abnormal outgrowths, leading to cancers and malignancy the product acts as an anti cancer product inactivating the outgrowths of cells and tissues opening a new window in cancer cure as it can regenerate the adult abnormal cells as well as embryonic cells within the body as well as replace them curing the abnormality completely.

### EXAMPLE 12

Product when administered to men and women in rectal prolapse as well as female genital prolapsed, it is seen that such medical condition is due to connective tissue disorder, once the product starts to regenerate the weak adult cells and connective tissues in the organ, through its ability of cell and connective tissue regeneration the medical conditions starts to neutralize. And over a course of time the adult cells and tissues regenerate completely or are replaced by new adult cells curing the medical condition completely.

### EXAMPLE 13

The product when administrated to women suffering from chronic white discharge it was seen that the medical condition neutralized regenerating the cells and tissues in the affected region and curing the problem over a course of time.

### EXAMPLE 14

The product when administrated to patients with puss from ear and the like it is seen that the weak adult cells and tissue start to regenerate and over a course of time normalize and function like a normal cell or tissue curing the medical condition.

### EXAMPLE 15

The product when administered to patient suffering from hysterias the product helps to stabilize the connective cells and tissues in the nervous system and cures the medical condition completely.

### EXAMPLE 16

The product when administered to children born abnormal where the brain development is not at par with the normally developed child, it was seen that during the growing age of such children the product regenerates the abnormal brain cells and connective tissues. The weakly developed brain cells start to perform normally; the connective cells and tissues start to receive the signals in and from the body at a normal rate, thus improving the medical condition slowly.

### EXAMPLE 17

The cell regenerative antioxidant percentage composition is as follows:
For 10 kgs of ingredients
BANANA FLOWER: 9.7 kg or 9700 gms or 97% by weight
CUCUMBER FRUIT: 0.25 kg or 250 gms or 2.5% by weight
CHICKPEA: 0.05 kg or 50 gms or 0.5% by weight
TOTAL: 10 kgs or 10,000 gms

Finally, it will be understood that this invention as described and/or illustrated within this specification is in no way limited to the preferred experimental combination of the ingredients and results as described herein by way of illustration. The cell regenerating antioxidant composition and the process for preparing the said composition made from commonly available flowers and fruits, which includes banana flowers, cucumber fruits and chickpea, the product repairs and regenerates the cells and the tissues within the body and the modified forms thereof, and the like lie within the scope of the invention. Those skilled in the art will appreciate that various modifications, additions, and substitutions are possible without departing from the scope and spirit of the invention, as set forth in the following claims.

1. A cell regenerating antioxidant composition comprising a mixture including flowers and fruits to regenerate and repair cells and tissues within a body.

2. A cell regenerating antioxidant composition as claimed in claim 1, wherein the flowers and the fruits comprising banana flowers, cucumber fruits and chickpea to regenerate and repair the cells and tissues within the body.

3. A cell regenerating antioxidant composition as claimed in claim 2, wherein by weight based on a total weight of a cell regenerating antioxidant product, the cell regenerating antioxidant product comprises 97% by weight of the banana flowers, 2.5% by weight of the cumber fruits and 0.5% by weight of the chickpea.

4. A cell regenerating antioxidant composition as claimed in claim 3, further comprising at least one of a pharmaceutically acceptable additive, carriers, excepient, and filler.

5. A cell regenerating antioxidant composition as claimed in claim 4, wherein the carries are selected from the group consisting of butter, milk, starch, sugar, cream, honey, vegetable oils, edible oils, fats, gums, glycols, water and other material working with white butter or yellow butter.

6. A cell regenerating antioxidant composition as claimed in claim 1, wherein each unit dosage of the composition comprises about 40 mg to 1000 mg or from 80 mg to 3000 mg of the mixture.

7. A cell regenerating antioxidant composition as claimed in claim 1, wherein the composition is in a form of powder, granules, pills, tablets, capsules, suspension, injection, syrup, tincture adhesive paste and the like.

8-11. (canceled)

12. A process for the preparation of cell regenerating antioxidant composition as claimed in claim 1, characterized in that:
a) the flowers and fruits as a whole are placed in aluminium or stainless steel vessel covered with a lid, the lid is filled with water on top to maintain the temperature below 100° C. once the heating starts, the vessel is heated and cooled for 5 to 7 days;
b) after 5-7 days the above mentioned ingredients of flowers and fruits shrink and leave a reactant mixture in liquid state which is drained and collected in another stainless steel container, after which the shrunk flowers and fruits are stirred (by way of rotating the semi solid substance upside down) in the vessel of step a;
c) to the above liquid reaction mixture in another container, chickpea is added and kept for 12-15 hours, then after 12-15 hours the chickpea is removed from the reactant mixture and allowed to germinate for next 24 to 36 hours;
d) to the vessel of step a above the liquid reactant mixture soaked with chickpea is reintroduced and after 12-15 hours the process of heating and cooling continues in the former vessel below 100° C. covered with a lid filled with water;
e) as the germination of the chickpea completes it is further reintroduced in the above vessel and processed below 100° C. for 3 to 4 days;
then the above reaction mixture is distilled (by the process of evaporation and condensation) for another 3-4 days with occasional stirring (by way of rotating the semi solid substance upside down), till the pulverized mixture of the cell regeneration antioxidant is obtained thereafter it is dried, powdered, sieved and filtered to get standardized extracts free of moisture.

13. A process—for the preparation of cell regenerating antioxidant composition as claimed in claim 12, wherein by weight based on a total weight of the cell regenerating antioxidant product, the cell regenerating antioxidant product comprises 97% by weight of banana flowers, 2.5% by weight of cumber fruits and 0.5% by weight of the chickpea.

14. (canceled)
